(19) United States
(12) Patent Application Publication
(10) Pub. No.: US 2025/0316363 A1
(43) Pub. Date: Oct. 9, 2025

(54) Method of adapting medical images includes adding a human anatomy diagram overlay to identify each image's position within the diagram and displaying a small version of the diagram within each image and saving the images in a standard known format.

(71) Applicants: Steven Troppoll, Carlisle, MA (US); Ralf Berger, Acton, MA (US)

(72) Inventors: Steven Troppoll, Carlisle, MA (US); Ralf Berger, Acton, MA (US)

(21) Appl. No.: 18/445,938

(22) Filed: Apr. 5, 2024

(51) Int. Cl. G16H 30/40; G16H 10/60; G16H 15/00; G16H 30/20

(52) U.S. Cl. CPC G16H 30/40; G16H 10/60; G16H 15/00; G16H 30/20

(57) ABSTRACT

A medical apparatus and method of using the same allowing a user to capture a medical image and add a human anatomy diagram overlay on the medical image to specify each image's position on its own version of the diagram and display a small version of the diagram on the image, incorporated by reference in their entirety herein U.S. Pat. No. 8,924,864 and U.S. Pat. No. 10,638,089.

A method of adapting medical images includes adding a human anatomy diagram overlay to identify each image's position within the diagram and displaying a small version of the diagram within each image and saving the images in a standard known format.

### ABSTRACT

A medical apparatus and method of using the same allowing a user to capture a medical image and add a human anatomy diagram overlay on the medical image to specify each image's position on its own version of the diagram and display a small version of the diagram on the image, incorporated by reference in their entirety herein U.S. Pat. No. 8,924,864 and U.S. Pat. No. 10,638,089.

### FIELD

The invention relates to an apparatus and method of use adapted for receiving an input medical image from a medical acquisition system and selecting the type of human anatomy diagram desired, displaying a human anatomy diagram as an overlay on a medical image, selecting the location to mark the area of concern for that image on the human anatomy diagram, saving the image with the overlay human anatomy diagram in any known standard image file format, (i.e. DICOM, MP4, PDF), and generating a report to include the images with overlay diagrams to be included in a patient's electronic medical record, patient report, or any medical report desired to be included with the patient information.

### Background

The colonoscopy procedure, where an endoscope is passed through the colon and images are captured, results in a report document that explains the results and includes the images and often a diagram showing the location of the images within the colon. The process of specifying the image location within the diagram has many issues as does the display within the report. This invention is a novel approach for specifying and displaying the image location within the diagram.

Typical report examples contain a diagram and series of images. The images contain a number overlay in a corner that can be used to identify the image location using the corresponding number in the diagram.

Prior art makes the numbering of many images with one diagram as in (FIG. 1), as commonly known by persons of ordinary skill in the art.

With many images and one diagram, the report generated quickly becomes cluttered as the number of images increases, trying to associate the image location and corresponding number in the one diagram. They become difficult to find and rearrange when trying to relocate them, or deciding to not include some of them during the generation of the report. For the reader, the challenge is to find the image and the corresponding image number in the diagram, which can become a big effort, especially if there are many images, or the diagram is on a different page than some or all of the images. The resulting report has the challenge of where to put the diagram and the images, especially with a large number of images that can be on more than one page.

This invention, in one embodiment, is to specify each image's location on its own version of the diagram and then display a very small version of that diagram on the image. Essentially, replacing the image number overlay with the overlay diagram itself (FIG. 2).

The software interface illustration looks like (FIG. 3). As you move through the images, you see only that specific image's location on the diagram. You have the choice to click on the diagram to specify the location. You can click somewhere else on the diagram to move it, or click on it to erase it. If you decide to delete an image from the study, the information is self-contained and does not affect any other diagrams.

This invention's report would look like (FIG. 4). The location of the image in the diagram is immediately: obvious. Rather than having to look from the image up to the diagram and locate the number in the diagram, it is directly with that image. If the report runs to multiple pages, this solution is not affected.

The preferred embodiment and modes of operation of the present invention have been described in the foregoing specification. The invention, which is intended to be protected herein, however, is not to be construed as limited to the particular embodiments disclosed, since these embodiments are to be regarded as illustrative rather than restrictive. Variations and changes may be made by others without departing from the spirit of the invention. Accordingly, it is expressly intended that all such variations and changes which fall within the spirit and scope of the claims be embraced thereby.

### BRIEF DESCRIPTION OF DRAWINGS

Other objects, features and advantages will occur to those skilled in the art from the following description of an embodiment and the accompanying drawings, in which:

FIG. 1. Shows prior art numbering of many images and one diagram

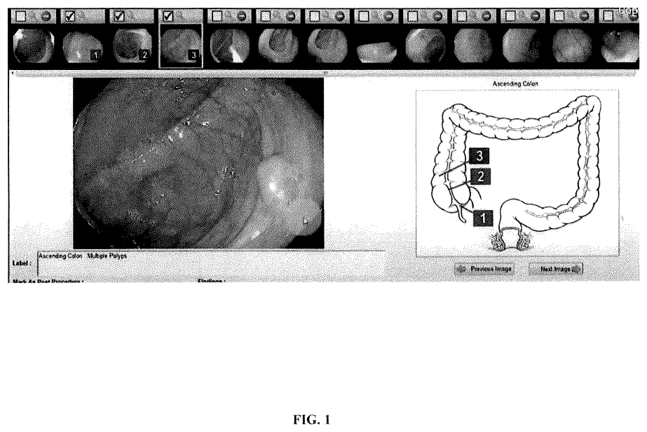

FIG. 2 Shows each image with their own version of the diagram

FIG. 3 Shows the software interface of the invention as a user moves through the images

FIG. 4 Shows the report generated by the invention

FIG. 5 Shows the overlay diagram with highlighted area of concern location of rectum

FIG. 6 Shows the overlay diagram with highlighted area of concern location of sigmoid colon

### DETAILED DESCRIPTION

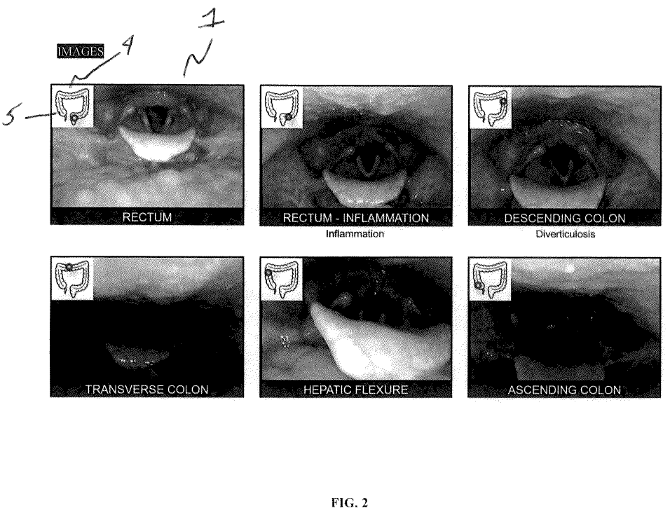

This invention, in one embodiment, is to specify each image's location 5 on its own version of the diagram 4 and then display a very small version of that diagram on the image 1, FIG. 2. Essentially, replacing the image number overlay with the overlay diagram 4 itself.

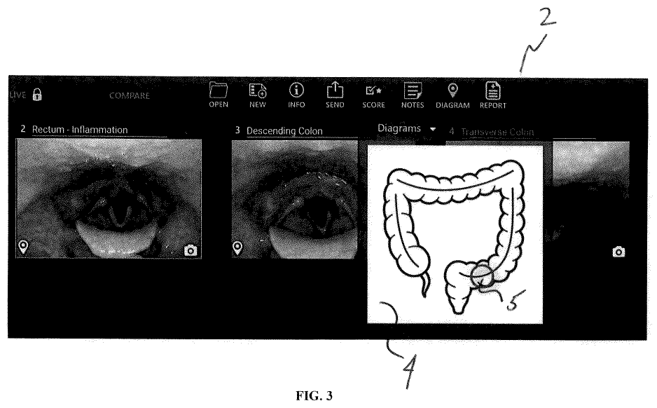

The software interface looks like FIG. 3. As you move through the images 2, you see only that specific image's location 5 on the diagram 4. You have the choice to click on the diagram to specify the location. You can click somewhere else on the diagram to move it, or click on it to erase it. If you decide to delete an image from the study, the information is self-contained and does not affect any other diagrams.

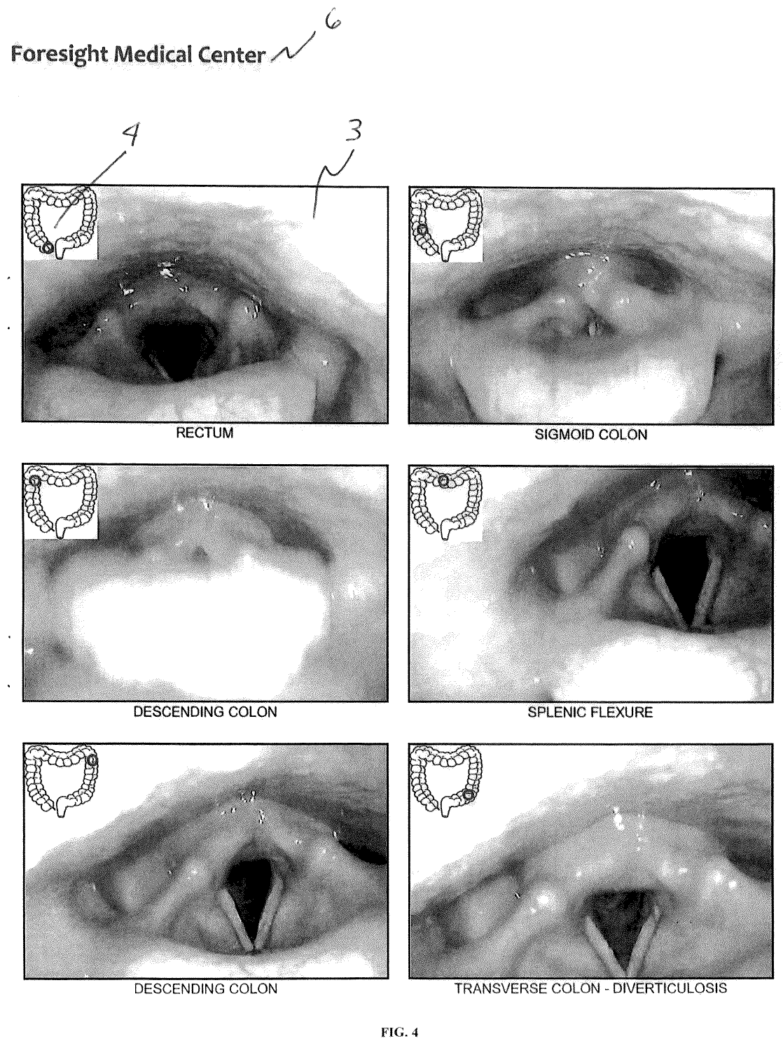

The generated report 6 would look like FIG. 4. The location 5 of the image 3 in the diagram 4 is immediately obvious. Rather than having to look from the image up to the diagram and locate the number in the diagram, it is directly with the image. If the report runs to multiple pages, this solution is not affected.

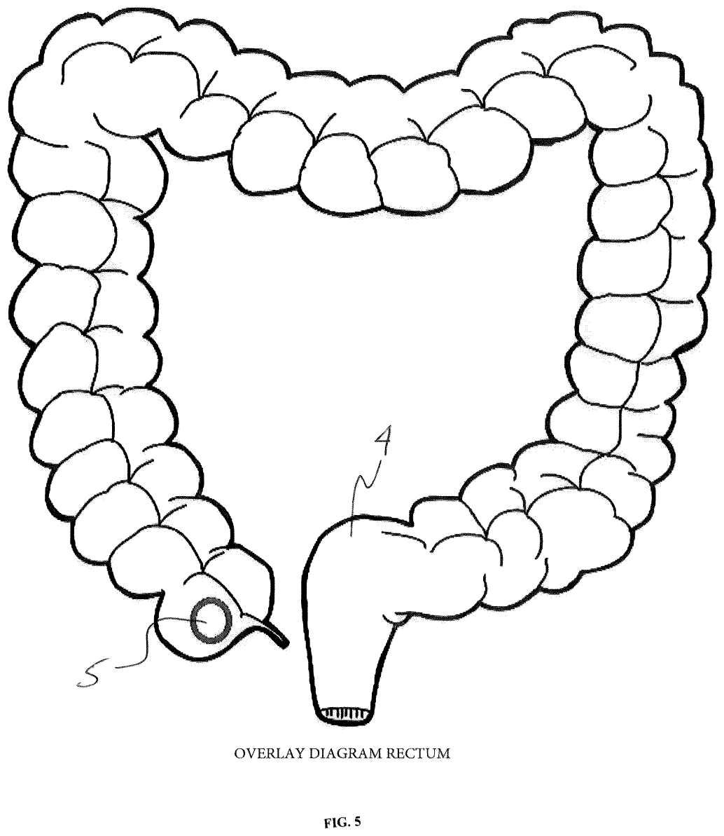

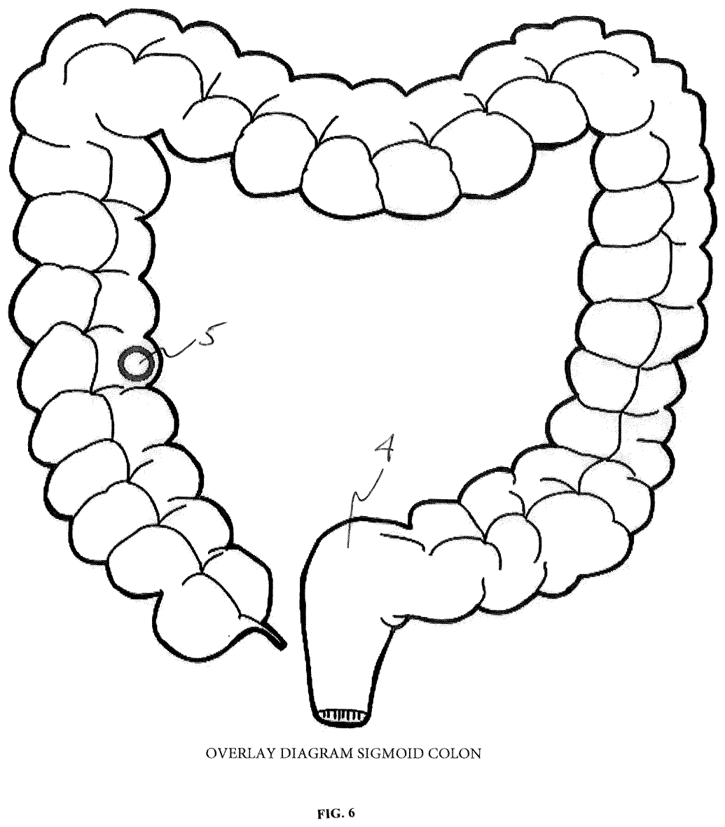

FIG. 5 and FIG. 6 are enlarged graphics of the overlay diagram 4 with the respective location 5 for each for easier viewing of the overlay diagram 4 and location 5. Labels 7 can be added to provide additional information about the anatomy diagram.

Anatomy of this invention is defined as any human body part, such as Esophagus, Large Intestine, Small Intestine, Colon, Ears, Nose, Throat, Lungs, Heart, Vagina, Urethra, bladder, Urinary Tract, Abdomen, or any body part that an Endoscope can enter the human body through its natural orifices or through an incision. These human body parts listed are meant to be examples for one skilled in the art, and not meant to be restrictive in any manner to a Medical Professional or Clinician that is skilled in the field of Endoscopy.

What is claimed is:

1. A method of displaying a human anatomy diagram as an overlay on a medical image specifying the area of concern location on the overlay human anatomy diagram,
said method comprising:
receiving an input medical image from a medical acquisition system;
selecting the type of human anatomy diagram desired;
selecting the location to mark the area of concern for that image on the human anatomy diagram
saving the image with the overlay human anatomy diagram in any known standard image file format, (i.e. DICOM, MP4, PDF),

2. A method of displaying a human anatomy diagram as an overlay on a medical image specifying the area of concern location on the overlay human anatomy diagram as in claim 1 wherein said diagram can be moved to any location on the medical image

3. A method of displaying a human anatomy diagram as an overlay on a medical image specifying the area of concern location on the overlay human anatomy diagram as in claim 1 wherein receiving one or more medical images

4. A method of displaying a human anatomy diagram as an overlay on a medical image specifying the area of concern location on the overlay human anatomy diagram as in claim 1 wherein said image being video image

5. A method of displaying a human anatomy diagram as an overlay on a medical image specifying the area of concern location on the overlay human anatomy diagram as in claim 1 wherein deleting an image from the study does not affect any of the other diagrams on the other images

6. A method of displaying a human anatomy diagram as an overlay on a medical image specifying the area of concern location on the overlay human anatomy diagram as in claim 1 wherein said image with overlay diagram is included in a patient's electronic medical record

7. A method of displaying a human anatomy diagram as an overlay on a medical image specifying the area of concern location on the overlay human anatomy diagram as in claim 1 wherein said image with overlay diagram is included in a patient report

8. A method of displaying a human anatomy diagram as an overlay on a medical image specifying the area of concern location on the overlay human anatomy diagram as in claim 1 wherein said overlay diagram can be any human anatomy diagram

9. A medical system apparatus allowing a user to capture a medical image and add a human anatomy diagram as an overlay on the medical image to specify the area of concern location on the overlay human anatomy diagram, comprising:
a medical image acquisition system computer adapted for acquisition of at least one medical image wherein signals are acquired from a transmitting medical device in the medical device's native resolution having a central processing unit, a display, at least one input device, a storage device and output device wherein the medical images are displayed on the output device, receiving an input medical image from a medical acquisition system; selecting the type of human anatomy diagram desired; selecting the location to mark the area of concern for that image on the human anatomy diagram and saving the image with the overlay human anatomy diagram in any known standard medical image file format, (i.e. DICOM, MP4, PDF), to be included in a patient's medical report, a patient electronic medical record, or any medical report desired to be included with the patient information
